(19) United States
(12) Patent Application Publication
(10) Pub. No.: US 2006/0160732 A1
(43) Pub. Date: Jul. 20, 2006

(54) Compositions and methods for inhibiting cell senescence and hyperproliferative disorders

(76) Inventor: Gary D. Aronson, Incline Village, NV (US)

Correspondence Address: PILLSBURY WINTHROP SHAW PITTMAN LLP, ATTENTION: DOCKETING DEPARTMENT, P.O BOX 10500, McLean, VA, 22102, US

(21) Appl. No.: 10/961,824

(22) Filed: Oct. 8, 2004

Related U.S. Application Data
(60) Provisional application No. 60/510,602, filed on Oct. 10, 2003.

(51) Int. Cl. A61K 38/16; C12Q 1/70; C07H 21/04; C07K 14/16

(52) U.S. Cl. 514/12; 435/5; 435/69.3; 435/456; 435/325; 530/350; 536/23.72

(57) ABSTRACT

The present disclosure provides compositions and methods for inhibiting cell and/or organismic senescence by treating conditions associated with aging and preventing undesirable cell proliferation. Compositions provided in the present disclosure include a transport agent attached to a therapeutic agent portion, wherein the transport agent portion has a role in transporting the composition across one or more biological membranes and the therapeutic agent portion prolongs cell life by effects on the proliferative capacity of a cell. In particular, the therapeutic agent includes a first region having telomerase activity and a second region having tumor suppressor activity.

### FIELD OF THE INVENTION

The present invention is directed to compositions and methods for inhibiting cell and/or organismic senescence. In particular, the present invention is directed to compositions and methods for inhibiting cell and/or organismic senescence by transporting a composition across biological membranes, wherein the composition includes a transport agent attached to a therapeutic agent. More specifically, the invention is directed to transport of a therapeutic agent into a cell, wherein the therapeutic agent may have an effect on a plurality of cellular processes including telomere length and cell proliferation. The present invention represents an improvement on alternative methods by simultaneously inhibiting and/or controlling hyperproliferative disorders while inhibiting cellular and organismic senescence.

### BACKGROUND OF THE INVENTION

With normal cellular aging, the rate of cell division slows and eventually stops, as observed in cultured human cells that reach growth arrest after dividing around 60 to 80 times. This loss of proliferative capacity is considered a hallmark of cellular senescence. In contrast, cancer cells often do not show cellular senescence and instead, exhibit undesirable proliferation. Treating cellular senescence should restore the ability of normal cells to divide without provoking undesirable cell proliferation.

Transport Agents

Many therapeutic agents that act on intracellular targets cannot easily cross biological membranes such as the plasma membrane, nuclear membrane, or organellar membranes. Transport agents can be conjugated to therapeutic agents to escort the resulting conjugate across biological membranes. Transport agents can also target a therapeutic agent to a specific intracellular location. Transport agents include the transit peptides or signal peptides that are normally used to target newly synthesized proteins to their cellular destination. Transport agents include protein transduction vehicles such as the antennapedia peptide, the Herpes simplex virus VP22 protein, or the HIV tat protein transduction domain. Transport agents also include high-molecular-weight polylysine polymers, or protein fragments such as arginine-rich sequences or highly basic guanidino-rich or amidino-rich polymers as described in U.S. Pat. No. 6,306,993. Transport agents are often cleaved after the conjugate has crossed a biological membrane, trapping the therapeutic agent inside the target cell or intracellular compartment.

Cellular Senescence

Cellular senescence can refer to a collection of events associated with cellular aging including metabolic, morphological, and genetic changes. Growth arrest, or the loss of ability to divide, is considered a hallmark of cellular senescence. Telomeres, and the proteins, nucleic acids, and metabolic processes associated with telomeres, are considered to have an important role in cellular senescence.

Telomeres are protein-DNA structures located at the ends of chromosomes. In most organisms, telomeric DNA consists of a tandem array of very simple sequences, e.g., human telomeric DNA consists of hundreds to thousands of tandem repeats of the sequence TTAGGG. During mitosis, chromosome replication requires the action of DNA polymerases that require an RNA primer and can proceed only in a 5′ to 3′ direction. Thus, RNA bound at the extreme 5′ ends of eukaryotic chromosomal DNA strands is removed during mitosis, leading to a progressive shortening of telomeres with each mitotic division. Shorter telomere lengths in human adults correlate with poor health and higher mortality rates. The length and integrity of telomeres appears related to entry of a cell into a senescent stage wherein it suffers loss of proliferative capacity. However, induced expression of telomerase in cells in culture allows cells to continue growing and dividing without becoming senescent. (Bodnar et al., 1998, Science 279:349-352) In addition, there appears to be a telomere position effect (TPE) involved in reversible silencing of genes located near telomeres (Baur et al., 2001, Science 292:2075-2077), which suggests that age-related telomere shortening results in de-repression of aging-related genes. In light of these observations, it has been proposed that telomeric shortening accounts for the phenomenon of cellular senescence (cell aging) of normal human somatic cells in vivo and in vitro (especially, in cell culture), and has important contributory effects on organismic aging, as. Thus, telomere length is considered not only a “mitotic clock” for a cell, but also a determinant of survival for a cell or an organism (an individual). Conversely, the ability of a cell to maintain or increase telomere length may allow a cell to escape senescence and continue to grow and divide. If these growth and division abilities are regulated, they are beneficial contributors to organismic growth and regeneration. If they are not appropriately regulated, they can contribute to cancer and other hyperproliferative disorders.

Regulation of Cell Proliferation

The control of cell proliferation is a complex process involving multiple interacting components. Whether a cell grows or not depends on the balance of the expression of negatively-acting and positively-acting growth regulatory genes. Negatively-acting growth regulatory genes are those that, when expressed in or provided to a cell, lead to suppression of cell growth. Positively-acting growth regulatory genes are those which, when expressed in or provided to a cell, stimulate its proliferation. Several negatively acting growth regulatory genes called tumor suppressor genes which have a negative effect on cell proliferation have been identified. These genes include the human retinoblastoma (Rb) gene and the p53 gene. Absence, damage, mutation or inactivation of some of these negative growth regulatory genes has been correlated with certain types of cancer, such that some of these genes are known as tumor suppressor genes.

For example, the p53 tumor suppressor gene/protein has a central role in suppressing abnormal cell proliferation and maintaining cell health. (Harris, 1993, Science, 262: 1980-1981) The p53 gene is located on chromosome 17, and was so named because the gene encodes a protein having a molecular weight of 53 KDa. The p53 protein acts, inter alia, by regulating DNA transcription, by detecting DNA mutations, and by preventing damaged (mutation-containing) cells from proliferating. The p53 protein has been called the “guardian of the genome” in recognition of its central role in maintaining cell health.

Mutations in the p53 gene can interfere with the ability of p53 protein to block abnormal cell growth, and p53 mutations are the most frequently observed genetic lesions in human cancers. In fact, p53 was first thought to be an oncogene because the first p53 clones that were isolated corresponded to mutant forms expressed in immortalized cell lines. Many mutant forms of p53 are oncogenic. It was observed that expression of mutant tumor-derived p53 immortalizes primary fibroblasts, and in combination with mutant ras, transforms these cells to a cancerous condition. In contrast, expression of normal or wild-type p53 functions as a tumor suppressor, and overexpression of normal p53 can inhibit the growth of various tumor cell lines and block the transforming activity of a variety of oncogenes. It has been observed that tumor-derived mutant p53 proteins do not bind to DNA in the same way as wild-type p53 proteins.

Retinoblastoma (Rb) protein is also known to play a key role in controlling normal cell proliferation and differentiation. Rb is believed to keep normal cells from dividing by maintaining them in the G1 or G0 phase of the cell cycle. Rb also binds to cellular proteins that regulate transcription. Rb is considered a tumor suppressor because abnormal growth of a cancer cell can result from inactivation of Rb protein. Inactivation can occur either due to mutation of the gene or due to inactivation of Rb protein by binding a viral oncoprotein encoded by an oncogenic tumor virus.

### DETAILED DESCRIPTION OF THE INVENTION

All patent applications, patents, and literature references referred to in this specification are hereby incorporated by reference in their entirety.

Compositions and methods are provided herein for inhibiting cell senescence. In particular, compositions and methods are provided for transporting a composition across biological membranes to inhibit cell senescence by treating conditions associated with aging and preventing undesirable cell proliferation. Methods are provided herein for inhibiting cell senescence by administering compositions of the present invention.

A composition in accordance with the present invention includes a transport agent portion attached to a therapeutic agent portion, wherein the transport agent portion has a role in transporting the composition across one or more biological membranes and the therapeutic agent portion has a role in inhibiting cell senescence. It will be understood that from henceforth, the term “a transport agent” refers to a transport agent portion that may include multiple transport agent molecules; likewise, the term “a therapeutic agent” refers to a therapeutic agent portion that may include multiple therapeutic agents or components thereof. A therapeutic agent in accordance with the present invention may include two, three, four, or more regions, where each region may have a different effect in a cell. In one embodiment, the therapeutic agent is a polypeptide having two regions, each region having a different effect in a cell. In a further embodiment, the therapeutic agent has a first region having telomerase activity and a second region having tumor suppressor activity, where the first region is directed to inhibiting cell senescence and the second region is directed to inhibiting undesirable cell proliferation. In another embodiment, the therapeutic agent is a nucleic acid encoding at least two expression products, wherein the products may be expressed separately and chemically conjugated, or may be expressed as a fusion protein, wherein each product may have a different effect in a cell. In another embodiment, the therapeutic agent is a complex that may include, but is not limited to, polypeptides, nucleic acids, ribonucleoproteins, polysaccharides, lipids, lipopolysaccharides, non-naturally-occurring molecules, synthetic molecules, and variants, derivatives, or analogs thereof. Optionally, the transport agent is cleaved in vivo after the composition is transported across one or more biological membranes.

In accordance with one aspect of the present invention, the therapeutic agent inhibits cell senescence by treating conditions associated with aging. In accordance with another aspect, administration of compositions including at least one therapeutic agent of the present invention inhibits cell senescence by treating conditions associated with aging. In one embodiment, cell senescence is inhibited by inhibiting loss of proliferative capacity. In another embodiment, cell senescence is inhibited by inhibiting at least one disease associated with cellular aging. In another embodiment, cell senescence is inhibited by stimulating at least one repair process. In yet another embodiment, cell senescence is inhibited by stimulating at least one cellular process. It is understood that a therapeutic agent in accordance with the present invention can have multiple effects on a cell, with the result that cell senescence is inhibited.

In accordance with another aspect of the present invention, the therapeutic agent inhibits undesirable cell proliferation. In accordance with another aspect, administration of compositions including at least one therapeutic agent of the present invention inhibits undesirable cell proliferation. In one embodiment, undesirable cell proliferation is inhibited by treating cancerous or precancerous conditions in cells. In another embodiment, undesirable cell proliferation is inhibited by preventing the development of cancerous or precancerous conditions in cells.

In accordance with another aspect, compositions of the present invention are administered to a cell, a collection of cells, a tissue, an organ, an organism, or an individual to introduce one or more therapeutic agents to inhibit cell senescence. Accordingly, compositions of the present invention are administered to a cell, a collection of cells, a tissue, an organ, an organism, or an individual to introduce one or more therapeutic agents to treat conditions associated with aging and to inhibit undesirable cell proliferation. In one embodiment, compositions of the present invention are administered to an organism or an individual to treat diseases and conditions associated with human aging. In another embodiment, compositions of the present invention are administered to an organism or an individual to extend the lifespan of the organism or individual.

In accordance with one aspect, the invention provides administration of compositions including at least one protein therapeutic agent. In accordance with another aspect, the invention provides administration of compositions including expression vectors that express therapeutic agents of the present invention in cells transformed with the expression vectors. In accordance with another aspect, the invention provides administration of nucleic acids, in particular DNA and/or RNA, that are expressed in cells that take up the nucleic acids and/or suppress the expression of other genes. It is understood that administration of one or more compositions of the present invention to a cell, a collection of cells, a tissue, an organ, an organism, or an individual exposes each cell to the one or more compositions of the invention, which may lead to different effects in each cell. A plurality of therapeutic agents may be administered in accordance with the methods of the present invention.

In accordance with another aspect, the therapeutic agent inhibits cell senescence in adult stem cells in culture. In accordance with yet another aspect, the therapeutic agent inhibits cell senescence in transplanted adult stem cells. It is understood the present invention provides that a therapeutic agent may have multiple effects in a stem cell, or multiple therapeutic agents may be administered to an adult stem cell to have multiple effects in a stem cell, to achieve the effect of inhibiting cell senescence. Accordingly, a cell, a collection of cells, a tissue, an organ, an organism, or an individual may be contacted with compositions of the present invention, to introduce one or more therapeutic agents to inhibit cell senescence in adult stem cells. In one embodiment, compositions of the present invention are administered to a collection of cells, a tissue, an organ, an organism, or an individual to introduce one or more therapeutic agents to treat conditions associated with adult stem cell and/or somatic cell aging and prevent undesirable proliferation of adult stem cells and/or somatic cells.

### DEFINITIONS

The terms “polypeptide” and “peptide” and “protein” are used herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation, such as conjugation with a labelling component. It is understood that the term “polypeptide” may refer to all or part of a protein. In certain embodiments, a polypeptide is a structural unit of a protein, some proteins consisting of one, some of several polypeptides.

It is understood that “nucleotide sequence” as used herein can refer to a polynucleotide molecule having a certain sequence (a defined linear arrangement of bases), and can also refer to the sequence itself and the information content of a sequence—i.e., the information contained in a defined linear arrangement of bases. Nucleotide sequences can be single stranded or double stranded depending on the particular embodiment.

The DNA or RNA for nucleotide sequences, nucleic acid constructs, expression vectors, or telomerase substrates of the present invention can be synthetic or may be derived from any suitable species. Synthetic DNA or RNA includes analogs and non-naturally-occurring nucleotides such as PNA or LNA. All that is required is that the DNA or RNA carry out the intended function in a prokaryotic or eukaryotic organism.

“Organism” refers to any life form belonging to any taxonomic kingdom.

“Individual” refers to any vertebrate, particularly a member of a mammalian species, and includes but is not limited to domestic animals, sport animals, and primates, including humans. It is understood that an individual would necessarily fall within the scope of the term organism.

The term “fusion protein” refers to a protein including regions in a different position than the sequence that occurs in nature. The regions may normally exist in separate proteins and are brought together in the fusion protein; or they may normally exist in the same protein but are placed in a new arrangement in the fusion protein. A fusion protein may be created, for example, by chemical synthesis, or by creating and translating a polynucleotide in which the peptide regions are encoded in the desired relationship. A fusion protein may contain two or more proteins or fragments of proteins directly contiguous to one another or separated by a linker or spacer composed of amino acids that may enhance the ability of each fragment to assume its pharmacologically desired or useful configuration(s).

The term “chimeric protein” refers to a protein with at least two adjacent polypeptides that are not naturally found adjacent. A chimeric protein can be formed by chemical conjugation of two or more polypeptides. Alternately a chimeric protein can be formed by expression of a nucleotide construct that includes adjacent nucleotide sequences that are not naturally found adjacent. A “chimeric molecule” or “chimeric composition” includes non-protein components attached to a chimeric protein.

The term “inhibit” or “inhibition” or “inhibiting” a condition refers generally to reducing, reversing, treating, ameliorating or preventing a condition, where the effect may be partial or total such that the condition may be partially or totally inhibited.

The term “inhibiting cell senescence” includes prevention of, or partial or total inhibition of, processes associated with cell senescence, especially loss of normal proliferative capacity.

The term “undesirable cell proliferation” refers to abnormal proliferation of cells in an organism or in culture, and includes any human or animal disease or disorder, affecting any one or any combination of organs, cavities or body parts, which is characterized by single or multiple local abnormal proliferations of cells, groups of cells or tissue(s), whether benign or malignant. “Undesirable cell proliferation” includes uncontrolled cell proliferation that may not cease, e.g., as seen in the proliferation of cancer cells with the immortal phenotype, or in immortalized cell culture lines. “Undesirable cell proliferation” includes uncontrolled cell proliferation that can cease, e.g., as seen in the proliferation of cells that give rise to benign tumors.

The term “inhibition” or “inhibiting” or “suppression” or “suppressing” undesirable cell proliferation includes partial or total inhibition of abnormal cell proliferation and also is meant to include decreases in the rate of abnormal proliferation of cells. The biologically inhibitory dose of the compositions of the present invention may be determined by assessing the effects of the composition on target malignant or abnormally proliferating cell growth in tissue culture, tumor growth in animals or cell culture, or any other method known to those of ordinary skill in the art.

For compositions disclosed herein, the dosage administered to a target cell, collection of cells, tissue, organ, organism, or individual, is dependent upon the age, clinical stage and extent of the disease or genetic predisposition of the target, as well as the weight, kind of concurrent treatment, if any, and nature of the pathological or malignant condition in the target. The effective delivery system useful in the method of the present invention may include an inert carrier such as saline, or phosphate-buffered saline, or any such carrier that does not interfere with the effectiveness of the compositions of the present invention. Pharmaceutical compositions may include a pharmaceutically acceptable excipient.

### Transport Agents

The present invention provides compositions that include a transport agent involved in transmembrane transport. In accordance with one aspect, a transport agent may be involved in permitting, facilitating, or enhancing transport across a biological membrane of composition that includes a therapeutic agent and a transport agent. Transport agents are expected to act by mechanisms including, but not limited to, enhancing the lipophilicity of a composition or binding of a composition to a recognition site on a molecule involved in internalizing the bound composition. Transport agents in accordance with the present invention may be proteins, polypeptides, peptides or peptide-like molecules, and may contain D or L amino acid residues, or non-naturally-occurring amino acids including hydroxy amino acids, N-methyl-amino acids, amino aldehydes, or synthetic amino acids, or any effective combination thereof. Transport agents may be signal peptides or transit peptides, or derivatives or fragments thereof, used for post-translational targeting of proteins across biological membranes to specific cellular destinations.

Transport agents may be lipopeptides, fatty acids, and basic polymers, e.g., tripalmitoyl-S-glycerylcysteil-seryl-serine, palmitic acid, polyarginine, octaarginine (Arg(8)) or oligoguanidino peptides, e.g., peptides having from 6 to 25 amino acid residues, at least 50% of which contain a guanidino or amidino sidechain moiety, and further having at least 6 contiguous guanidino and/or amidino sidechain moieties, as disclosed in U.S. Pat. No. 6,306,993. Transport agents may be arginine-rich peptides including RNA-binding peptides derived from viral proteins, e.g., HIV-1 Rev, flock house virus coat proteins, or DNA binding segments of leucine zipper proteins or arginine-rich transcription factors, e.g. yeast transcription factor GCN4.

Transport agents include protein transduction vehicles that promote protein entry into cells, e.g. antennapedia peptide, Herpes simplex virus VP22 protein, or trans-activating transduction (tat) proteins (Ford et al., 2001, Gene Therapy 8:1-4). In one embodiment, Drosophila antennapedia residues 43-58 is the transport agent. In another embodiment, purified human immunodeficiency virus type-1 (HIV) tat polypeptide is the transport agent. The entire HIV tat protein (86 amino acids) or any effective polypeptide fragment thereof can be used. In one embodiment, an arginine-rich region of HIV tat, e.g. amino acid residues 48-60, is used as a transport agent. In another embodiment, arginine-substituted HIV tat is the transport agent. In another embodiment, equine infectious anemia virus (EIAV) tat protein, or a fragment thereof, is used as a transport agent.

In one embodiment, the transport agent is Herpes simplex type 1 virus (HSV) structural protein VP22 protein or an effective polypeptide fragment thereof, especially the 34-amino acid C-terminal sequence. The herpesviral HSV-1 virion protein VP22 possesses an unusual intercellular trafficking mechanism, as the protein can efficiently transport itself through the membrane of cells via a non-classical Golgi-independent mechanism. (WO 97/05265; Elliott & O'Hare, 1997, Cell 88:223-233). When fused to a variety of other proteins the VP22 protein can transport the fused proteins across cell membranes thus carrying the attached proteins into the nucleus. VP22-fused proteins have been shown to retain biological activity and to deliver this activity directly into the exposed cell in a highly efficient manner. VP22-fusion protein transport capability has recently been demonstrated for a variety of different proteins including: green fluorescent protein (GFP), a 27 KDa fluorescent marker protein, (Elliott & O'Hare, 1999, Gene Therapy 6:149-151, 1999); p53, a 53 KDa cell cycle regulatory protein, (Phelan et al., 1998, Nature Biotechnology 16:440-443); thymidine kinase (TK), the 52 KDa enzyme serving as the converting enzyme in the pro-drug suicide protein combination routinely used in gene therapy trials; (Dilber et al., 1999, Gene Therapy 6:12-21), and beta-galactosidase, the 116 KDa bacterial enzyme widely employed as a reporter protein in gene expression studies (e.g., Invitrogen products). In the studies cited supra, chimeric VP22 fusion proteins were efficiently transported into fusion-protein-exposed cells and demonstrated the biological effects associated with each coupled protein.

Other suitable transport agents include but are not limited to: bacterial hemolysins or “blending agents” such as alamethicin or sulfhydryl activated lysins; cell entry components of bacterial toxins such as Pseudomonas exotoxin, tetanus toxin, ricin toxin and diphtheria toxin; proteins which are viral receptors, cell receptors or cell ligands for specific receptors that are internalized and cross mammalian cell membranes via specific interaction with cell surface receptors; immunogens including bacterial immunogens, parasitic immunogens, viral immunogens, immunoglobulins, or cytokines.

Various other proteins have the capability to permeate cellular membranes by the addition of a membrane-translocating sequence (MTS) (Rojas et al., 1998, Nature Biotechnology 16:370-375). In one embodiment, the MTS hydrophobic region (h-region) is used to deliver various peptides and proteins (cargo) across cell membranes in a nondestructive manner.

Optionally, a transport agent may be cleaved or removed from a therapeutic agent of the present invention after the composition has been transported across a biological membrane.

In accordance with one aspect, transport agents can be attached to therapeutic agents by recombinant means, preferably by fusion of at least one nucleotide sequence encoding a transport agent and at least one nucleotide sequence encoding a therapeutic agent to form a construct. Generally, a fused construct is part of, or is cloned into, an expression vector that is expressed in a suitable host using standard techniques of recombinant DNA technology, e.g. as disclosed in Sambrook et al., Molecular Cloning, 2^{nd} Edition, Cold Spring Harbor Laboratories, 1989. Suitable hosts include prokaryotic hosts such as bacteria, and eukaryotic hosts such as yeasts, insect cells, or mammalian cells. The design of an expression vector and selection of a suitable host can be carried out by one of skill in the art, and may be influenced by factors including post-translational processing of the expression product. Thus, nucleotide sequences encoding compositions used in the present invention, operatively linked to regulatory sequences, may be constructed and introduced into appropriate expression systems using conventional recombinant DNA techniques. The resulting fusion protein(s) may then be purified and tested for the capacity to enter target cells and inhibit cell senescence in accordance with the present invention.

In accordance with another aspect, a transport agent may be attached to a therapeutic agent by chemical (i.e., non-recombinant) means. A transport agent may be directly attached to a therapeutic agent, or may be attached via a linker. Optionally, a linker may contain a cleavage site. Chemical attachment of a transport agent to a therapeutic agent may be effected by any means which produces a bond between the two molecules, where the bond can withstand the conditions used, and which does not alter the activity or function of either molecule. Many chemical cross-linking agents are known and may be used to join a transport agent to a therapeutic agent. Suitable intermolecular cross-linking agents include, e.g., succinimidyl 3-(2-pyridyldithio)propionate (SPDP) or N,N′-(1,2-phenylene)bismaleimide, which are highly specific for sulfhydryl groups and form irreversible linkages; N,N′-ethylene-bis-(iodoacetamide) (specific for sulfhydryl); and 1,5-difluoro-2,4-dinitrobenzene (forming irreversible linkages with tyrosine and amino groups). Other agents include p,p′-difluoro-m,m′-dinitrodiphenylsulfone (forming irreversible linkages with amino and phenolic groups); dimethyl adipimidate (specific for amino groups); hexamethylenediisocyanate (specific for amino groups); disdiazobenzidine (specific for tyrosine and histidine); succinimidyl 4-(N-maleimidomethyl)cyclohexane-1-carboxylate (SMCC); m-maleimidobenzoyl-N-hydroxysuccinimide ester (MBS); and succinimide 4-(p-maleimidophenyl)butyrate (SMPB). The succinimidyl group of these cross-linkers reacts with a primary amine, and the thiol-reactive maleimide reacts with the thiol of a cysteine residue. See, Means and Feeney, Chemical Modification of Proteins, Holden-Day, 39-43, 1974; and Wong, Chemistry of Protein Conjugation and Cross-Linking, CRC Press, 1971. All the cross-linking agents discussed herein are commercially available and detailed instructions for their use are available from the suppliers.

In accordance with another aspect, a composition of the present invention may include multiple transport agents. Accordingly, a composition may include a transport agent directed at transporting the composition across the plasma membrane of a target cell and at least one additional transport agent directed at intracellular targeting of a therapeutic agent. In one embodiment, a transport agent involved in intracellular targeting is a nuclear localization signal involved in delivering a therapeutic agent to the nucleus. A variety of nuclear localization sequences are known in the art that can direct proteins to the cell nucleus, for example as disclosed by Dignwall et al. (1987, EMBO J 8:69-71).

It is understood that one of skill in the art can select and test transport agents for their ability to permit, facilitate, or enhance transport of a composition of the present invention across one or more biological membranes. One of skill in the art can modify transport agents and test modified transport agents. Fragments, variants, or derivatives of transport agents can likewise be tested to identify suitable transport agents. Transport agents can be tested, and their suitability determined, using methods that are well-known in the art, e.g., as disclosed in U.S. Pat. No. 6,306,993.

### Therapeutic Agents

The present invention provides a composition that is transported across at least one biological membrane, wherein the composition includes a transport agent and a therapeutic agent. It is understood that a composition of the present invention is directed to inhibiting cell senescence by a mechanism that includes the transport of the composition across at least one biological membrane and the action of the therapeutic agent inside the cell. A therapeutic agent in accordance with the present invention is directed to inhibiting cell senescence. A therapeutic agent in accordance with the present invention may have effects on one, two, or a plurality of cell processes to inhibit cell senescence. A therapeutic agent in accordance with the present invention is directed to treating conditions associated with cell aging and preventing undesirable proliferation. In particular, a therapeutic agent of the present invention is directed to inhibiting cell senescence by increasing telomerase activity with the aim to increase cell proliferative capacity, while also preventing the undesirable cell proliferation seen in cancerous or pre-cancerous conditions.

In accordance with one aspect, a therapeutic agent is directed to inhibiting cell senescence by restoring the ability of a cell to divide. In accordance with another aspect, a therapeutic agent is directed to stimulating cell growth. In one embodiment, a therapeutic agent treats cellular senescence by increasing telomere length. In another embodiment, a therapeutic agent inhibits the telomere position effect (TPE) on gene expression. In another embodiment, a therapeutic agent inhibits age-related effects on telomeres. In yet another embodiment, a therapeutic agent prevents age-related telomere shortening. In another embodiment, a therapeutic agent reverses age-related telomere shortening.

In accordance with another aspect, a therapeutic agent is directed to inhibiting undesirable cell proliferation. In one embodiment, a therapeutic agent treats cancer or pre-cancerous conditions that lead to undesirable cell proliferation. In another embodiment, a therapeutic agent prevents cancer or pre-cancerous conditions that lead to undesirable cell proliferation. In another embodiment, a therapeutic agent reverses cancer or pre-cancerous conditions that lead to undesirable cell proliferation.

In accordance with another aspect, a therapeutic agent of the present invention is directed to inhibiting cell senescence by stimulating cell growth and inhibiting undesirable cell proliferation. In accordance with another aspect, a therapeutic agent is directed to inhibiting cell senescence by inhibiting age-related effects on telomeres and by inhibiting cancer or pre-cancerous conditions. In accordance with yet another aspect, a therapeutic agent is directed to increasing telomere length and inhibiting oncogene-mediated undesirable cell proliferation. In one embodiment, a therapeutic agent of the present invention includes telomerase and normal p53 protein. In another embodiment, a therapeutic agent of the present invention includes telomerase and normal Rb protein. In yet another embodiment, a therapeutic agent of the present invention includes telomerase, normal p53 protein, and normal Rb protein. Alternately, a therapeutic agent of the present invention includes a nucleic acid construct encoding any or all of: telomerase; normal p53 protein; normal Rb protein. In other embodiment, modified versions of p53 and/or Rb proteins may be used instead of, and/or in addition to normal p53 and/or Rb proteins, where such modifications confer pharmacological advantages including, but not limited to, modified half-life, increased potency and/or efficacy and/or decreased toxicity and/or immunogenicity.

Increasing Telomere Length

In accordance with one aspect, a therapeutic agent of the present invention includes at least one component having an effect on telomere length. Accordingly, a therapeutic agent includes at least one component having an effect on the activity of telomerase or related enzymes.

Telomerase is a telomere-specific DNA polymerase which is involved in maintaining telomere length and can also be used to restore length to shortened telomeres. Telomerase is a ribonucleoprotein (RNP) that uses a portion of its RNA moiety as a template for telomere repeat DNA synthesis. For example, human telomerase adds repeated units of TTAGGG to the ends of telomeres. The catalytically active subunit of telomerase is telomerase reverse transcriptase (TRT, also known as TERT), encoded by the TERT gene. As used herein, “telomerase” refers generally to a molecule having telomerase activity, and includes telomerase ribonucleoprotein, TRT, and any effective variant or fragment of telomerase ribonucleoprotein or TRT having telomerase activity suitable for use in a therapeutic agent of the present invention.

In accordance with one aspect of the invention, telomerase is suitable for use in a therapeutic agent. Telomerase is active in germline cells but is not expressed in most normal somatic tissues. When cells do not express telomerase reverse transcriptase (TRT), then telomerase activity is low or absent, even when the template RNA component is being expressed. Bodnar et al. (1999, Science 279:349-352) showed that telomerase activity can be reconstituted in human cells by transient expression of human TRT (hTRT), with the result that telomerase-expressing cells have elongated telomeres, divide vigorously, and show reduced activity of an age-related enzymatic marker. In contrast, telomerase-negative (control) cells exhibit telomere shortening and cellular senescence. Notably, the telomerase-expressing cells exceeded their normal lifespan by at least 20 doublings, thus establishing a causal relationship between telomere shortening and in vitro cellular senescence. Without wishing to be limited by this theory, it is expected that introduction of telomerase, or some effective fragment thereof, into cells can prolong cell life by preventing or reversing cellular senescence, e.g., by increasing the proliferative capacity of a cell (See, U.S. Pat. No. 6,475,789).

In accordance with one aspect of the present invention, the therapeutic agent includes a region having telomerase activity. In one embodiment, the therapeutic agent includes telomerase. In one embodiment, the therapeutic agent includes a telomerase ribonucleoprotein complex. In another embodiment, the therapeutic includes telomerase reverse transcriptase (TRT, the catalytic subunit of telomerase). In another embodiment, the therapeutic agent includes an effective variant or fragment of telomerase. In another embodiment, the therapeutic agent includes an effective variant or fragment of TRT. In accordance with another aspect, the therapeutic agent includes a component that enhances telomerase activity or effectiveness. Telomerase may be human telomerase, and telomerase reverse transcriptase may be human telomerase reverse transcriptase (hTRT).

In accordance with another aspect, the therapeutic agent of the present invention includes at least one component having an effect on telomerase expression. In one embodiment, the therapeutic agent includes a nucleotide sequence encoding telomerase reverse transcriptase (TRT) or effective variant or fragment thereof. In another embodiment, the therapeutic agent includes a composition that alters telomerase expression. In another embodiment, the therapeutic agent includes a nucleotide sequence encoding a product that alters telomerase expression.

It is understood that one of skill in the art can determine telomerase activity and telomere length by methods known in the art, e.g., as described in U.S. Pat. Nos. 6,194,206; 6,337,200; 5,707,795; and 6,475,789. Likewise, it is understood that one of skill in the art can prepare therapeutic agents in accordance with the present invention, wherein the therapeutic agents include telomerase, TRT, an effective variant or fragment of telomerase or TRT, or a component having an effect on telomerase expression or activity.

Compositions and methods suitable for use in therapeutic agents of the present invention include but are not limited to: introducing into the cell an effective amount of a human telomerase reverse transcriptase (hTRT) polypeptide, or introducing a nucleic acid encoding hTRT into a cell, as disclosed in U.S. Pat. No. 6,475,789; culturing cells in the presence of an oligonucleotide substrate for telomerase, disclosed in U.S. Pat. No. 5,686,306; use of catalytically active human telomerase reverse transcriptase (hTRT) variants disclosed in U.S. Pat. No. 6,337,200; modulating expression of TRT by a mechanism that appears to be distinct from telomerase, disclosed in U.S. Pat. No. 6,331,399; providing heterogeneous ribonucleoprotein core protein A1 (hnRNP A1) and derivatives, disclosed in U.S. Pat. No. 6,294,332; or antisense modulation of hnRNP A1, disclosed in U.S. Pat. No. 6,165,789.

Inhibiting Undesirable Cell Proliferation

Compositions and methods of the present invention are directed to inhibiting cell senescence by restoring normal cell proliferative capacity, while also inhibiting undesirable cell proliferation. In accordance with one aspect, compositions and methods of the present invention are directed to inhibiting cell senescence by increasing telomerase activity or telomere length in order to restore or increase normal cell proliferative capacity, while also inhibiting the undesirable cell proliferation seen in cancer, pre-cancerous conditions, or non-cancerous hyperproliferative disorders. Telomerase activity is detected in immortal cell lines and a diverse set of tumor tissues, but is not detected in normal somatic cell cultures or normal tissues adjacent to a tumor (See, U.S. Pat. Nos. 5,629,154; 5,489,508; 5,648,215; and 5,639,613). It has been observed that lack of telomerase expression seems to curb growth of rapidly proliferating cells, whereas an increase in telomerase permits indefinite proliferation. For example, expression of telomerase in conjunction with expression of simian virus 40 large T oncoprotein and an oncogenic allele of H-ras has been shown to promote tumorigenic conversion of normal human cells (Hahn et al., 1999, Nature 400:464-468). It has been hypothesized that reactivation of telomerase is necessary to the undesirable cell proliferation seen in many tumors, because normal cells and early-stage carcinomas have little or no telomerase activity while late-stage carcinomas often have high telomerase activity. Accordingly, the present invention provides therapeutic agents that include at least one component directed to enhancing telomere length and at least one component directed to inhibiting undesirable cell proliferation.

In accordance with one aspect of the present invention, the therapeutic agent has a region having tumor suppressor activity. Without wishing to be limited by this theory, it is proposed that a therapeutic agent having tumor suppressor activity can inhibit undesirable cell proliferation. Accordingly, such a therapeutic agent can be used to inhibit undesirable proliferation by treating cancerous or pre-cancerous conditions in a cell or by inhibiting the development of cancerous or pre-cancerous conditions. Likewise, such a therapeutic agent can be used to inhibit undesirable proliferation by treating non-cancerous hyperproliferative disorders including, but not limited to, benign tumors, macular degeneration or diabetic retinopathy.

Genes and gene products that regulate cell proliferation are suitable for use in therapeutic agents of the present invention. Tumor suppressor genes are generally considered to be genes that function to suppress abnormal (undesirable) cell proliferation when the genes are in their “natural” or “normal” or “wild type” state. Damaged or mutant alleles of the same tumor suppressor gene(s) have an opposite effect and do not suppress abnormal cell proliferation. These genes are sometimes known as “anti-oncogenes” when their wild type or normal gene products have tumor suppressor activity and their mutant or inactivated gene products have the opposite effect. A cancer phenotype, including tumorigenesis, can result from damage, mutation, or inactivation of “anti-oncogene”-type tumor suppressor genes. Thus, nucleotide sequences encoding wild type tumor suppressor genes, or proteins encoded by wild type tumor suppressor genes, are suitable for use in therapeutic agents of the present invention. Other proteins (e.g., certain receptors or kinase inhibitors) are known to have tumor suppressor activity, but these proteins and the genes encoding them do not act like “anti-oncogenes,” i.e., these proteins and the genes encoding them do not have an opposite effect when damaged or mutant. Nucleotide sequences encoding tumor suppressor genes, or proteins encoded by tumor suppressor genes, are suitable for use in therapeutic agents of the present invention. Nucleic acids, polypeptides, or other compounds that modulate the activity of tumor suppressor genes or proteins are likewise suitable for use in therapeutic agents of the present invention.

Accordingly, components directed to having an effect on the activity of DNA-binding transcription factors such as p53, or transcription regulators such as retinoblastoma (Rb), or protein kinase inhibitors such as p16 are suitable for use in therapeutic agents of the present invention. The role of Rb as a tumor suppressor protein in cell-cycle control is believed to be similar to that of p53, but whereas p53 is generally believed to be responsive to environmental cues such as DNA damage, the Rb protein is apparently involved in coordinating cell growth with the exogenous stimuli that normally persuade a cell to cease proliferating. Accordingly, therapeutic agents of the present invention can be selected to include different tumor suppressors that act through different pathways to achieve a desired effect when used as provided herein.

It has been observed that re-introduction of wild-type or “normal” cDNA of RB-1 or p53 into a cell can partially restore normal growth regulation, as the re-introduced normal genes induce growth arrest or retardation in many different tumor cell types. It should be noted that the growth suppression effect of the Rb gene is not restricted to tumor cells. Normal cells which have two copies of the Rb gene can be growth-arrested or retarded by the introduction of extra copies of the normal Rb gene under certain growth conditions. Likewise, the ability of wild type or normal p53 protein to suppress the growth of noncancerous cells is well documented. Thus, the step(s) controlled by Rb and p53 may not directly affect the tumorigenic phenotype, but rather, may affect the steps that control the growth of tumor and normal cells alike.

It is understood that a skilled artisan can determine the effectiveness of a therapeutic agent to inhibit undesirable cell proliferation by using methods known in the art. Methods for determining the effectiveness of a tumor suppressor are particularly useful. One of skill in the art can administer a therapeutic agent and observe the effect on cell proliferation by measuring growth rate or colony formation in soft agar, or measuring tumor cell phenotype, or monitoring thymidine incorporation, or determining drug sensitivity, or by observing the tumorigenicity of cells that have been treated with therapeutic agent and placed in an experimental animal, e.g., as disclosed in U.S. Pat. No. 6,194,547. One of skill in the art can likewise measure the effectiveness of a therapeutic agent to inhibit undesirable cell proliferation by using or adapting methods for treatment of inappropriate or pathological cell growth in cancerous cell proliferative diseases and/or non-cancerous hyperproliferative disorders, by administering mutated growth suppressor gene and/or gene products, e.g., as disclosed in U.S. Pat. Nos. 6,200,801 and 5,969,120.

p53 Protein

In one embodiment, a therapeutic agent of the present invention includes a region that has an effect on p53 protein activity or expression or both. In one embodiment, a therapeutic agent of the present invention includes p53 protein, or an active fragment or variant thereof, having tumor suppressor activity. In another embodiment, a therapeutic agent includes a region that has an effect on p53 protein tumor suppressor activity. It is understood that the term “p53” or “p53 protein” or “normal p53 protein” in accordance with the present invention encompasses any member of the p53 protein family, any active fragment or variant thereof, and genes encoding the same, having tumor suppressor activity. One of skill in the art can determine whether a protein belongs to the p53 family based on features described in scientific literature. Without wishing to be limited by this theory, it is expected that compositions and methods directed to having an effect on p53 protein tumor suppressor activity will inhibit undesirable cell proliferation, as it was has been observed that enhanced p53 function leads to arrest of cell proliferation or to cell death.

In one embodiment, the therapeutic agent includes normal p53 protein. In another embodiment, the therapeutic includes an effective fragment or variant of normal p53. In one embodiment, the therapeutic agent includes p53 mutated to remain in active form, e.g., as described in U.S. Pat. Nos. 6,200,810 and 5,969,120. In another embodiment the therapeutic agent includes wild-type (normal) or mutant p53 protein, including wild-type p53 that suppresses neoplastic properties of cells transformed with vectors expressing the wild-type p53, wild-type p53 that is dominant to mutated p53, or mutant p53 that give a growth advantage to cells transformed with vectors expressing the mutant p53, e.g., as disclosed in U.S. Pat. No. 5,532,220. In another embodiment, the therapeutic agent includes a p53 isoform capable of inhibiting undesirable cell proliferation. In one embodiment, the therapeutic agent includes a p53 isoform with a C-terminal portion removed, e.g., a C-terminal-truncated 35 kDa (p35) isoform of p53 as described in U.S. Pat. No. 6,294,384. In another embodiment, the therapeutic agent includes a normal p53 protein and a p53 isoform with C-terminal portion removed, where the two proteins are chemically linked or expressed as a fusion protein. In another embodiment, the therapeutic agent includes a normal p53 protein and a normal p63 protein, where the two proteins are chemically linked or expressed as a fusion protein. In another embodiment, the therapeutic agent includes a normal p53 protein and a normal p73 protein, where the two proteins are chemically linked or expressed as a fusion protein. It is understood that one of skill in the art can test members of the p53 protein family, p53 fragments, variants, isoforms, or mutants to identify p53-related molecules having the desired effect when used in a therapeutic agent in accordance with the present invention. Likewise, it is understood that one of skill in the art can test nucleic acids encoding p53 fragments, variants, isoforms or mutants to identify nucleic acids encoding p53-related molecules having the desired activity when used in a therapeutic agent in accordance with the present invention. Preparing and testing p53-related molecules can be carried out using methods known in the art.

In accordance with another aspect, a therapeutic agent includes a component directed at increasing cell proliferative capacity and another component directed at inhibiting undesirable cell proliferation. In one embodiment, a therapeutic agent in accordance with the present invention contains telomerase and p53. In one embodiment, a therapeutic agent in accordance with the present invention contains TRT and p53. In another embodiment, a therapeutic agent in accordance with the present invention contains a nucleotide sequence encoding telomerase and p53, wherein expression of the nucleotide sequence generates a fusion protein including telomerase and p53. In another embodiment, a therapeutic agent contains a nucleotide sequence encoding TRT and p53, wherein expression of the nucleotide sequence generates a fusion protein including TRT and p53. It is understood one of skill in the art can test fragments, variants, isoforms or mutants of telomerase, and fragments, variants, isoforms or mutants of p53, to identify those molecules having the desired activity when used in a therapeutic agent according to the present invention.

Retinoblastoma Protein

In one embodiment, the therapeutic agent includes a region directed to having an effect on retinoblastoma protein activity or expression or both. In one embodiment, the therapeutic agent includes Rb protein having tumor suppressor activity (normal Rb). In another embodiment, the therapeutic agent includes a molecule that regulates retinoblastoma protein tumor suppressor activity. In yet another embodiment, the therapeutic agent according to the present invention contains a retinoblastoma gene (Rb gene) or retinoblastoma protein (Rb protein) mutated to remain in active form, e.g., as described in U.S. Pat. Nos. 6,200,810 and 5,969,120 which describe treatment of inappropriate or pathological cell growth by administering a synthetic mutated Rb gene encoding a functionally active Rb protein mutated to alter phosphorylation sites. In another embodiment, the therapeutic agent according to the present invention contains a wild-type (normal) Rb gene or protein that supplies functionally active Rb protein to cells lacking active Rb protein, e.g., as described in U.S. Pat. No. 5,858,771.

Rb is known to play a key role in controlling normal cell proliferation and differentiation. Rb is believed to keep normal cells from dividing by maintaining them in the G1 or G0 phase of the cell cycle. Rb also binds to cellular proteins that regulate transcription. Rb is an attractive target for controlling cell growth by a mechanism that includes blocking the action of Rb in cells and tissues that normally do not grow because of the action of Rb. The human retinoblastoma gene, RB-1, is a tumor suppressor gene in which the absence of both alleles of the gene in a cell, or the inhibition of the expression of the gene or its gene product, will lead to neoplastic or abnormal cell proliferation. At the molecular level, loss or inactivation of both alleles of RB-1 is involved in the clinical manifestation of tumors such as retinoblastoma, and clinically related tumors such as osteosarcomas, fibrosarcomas, soft tissue sarcomas and melanomas. In addition, loss of the function of RB-1 has also been associated with other types of primary cancer such as primary small cell lung carcinoma, bladder carcinoma, breast carcinomas, cervical carcinomas and prostate carcinomas. Re-introduction of a wild-type cDNA of RB-1 can partially restore normal growth regulation. Designation of the Rb gene as a tumor suppressor gene stemmed from the observation that inactivation of an allele of the Rb gene is a predisposing factor for development of cancer. However, the growth suppression effect of the Rb gene is not restricted to tumor cells. Normal cells which have two copies of Rb can be growth-arrested or retarded by the introduction of extra copies of the Rb gene under certain growth conditions. Thus, the step controlled by Rb may not directly affect the tumorigenic phenotype, but rather, may affect the steps that control the growth of tumor and normal cells alike.

Rb is also known as a tumor suppressor since abnormal growth of a cancer cell can result from inactivation of Rb protein. Inactivation can occur either due to mutation of the gene or inactivation of Rb protein by binding a viral oncoprotein encoded by an oncogenic tumor virus. The region known as the Rb pocket appears to be critical for the growth controlling function. U.S. Pat. No. 6,468,985 discloses Rb-interacting zinc finger (RIZ) proteins, and in particular the PR domain of RIZ, and use of the protein to bind Rb, which is involved in regulating cell proliferation.

In accordance with another aspect, a therapeutic agent includes a region directed at increasing cell proliferative capacity and another region directed to inhibiting undesirable cell proliferation. In one embodiment, a therapeutic agent in accordance with the present invention contains telomerase and Rb. In one embodiment, a therapeutic agent in accordance with the present invention contains TRT and Rb. In another embodiment, a therapeutic agent in accordance with the present invention contains a nucleotide sequence encoding telomerase and Rb, wherein expression of the nucleotide sequence generates a fusion protein including telomerase and Rb. In another embodiment, a therapeutic agent contains a nucleotide sequence encoding TRT and Rb, wherein expression of the nucleotide sequence generates a fusion protein including TRT and Rb. It is understood one of skill in the art can test fragments, variants, isoforms or mutants of telomerase, and fragments, variants, isoforms or mutants of Rb, to identify those molecules having the desired activity when used in a therapeutic agent according to the present invention.

In accordance with another aspect of the invention, the therapeutic agent contains a combination of both Rb and p53, or nucleic acids encoding these proteins. It is known that p53 and Rb regulate cell proliferation through different pathways, such that a therapeutic agent having a combination of Rb and p53 would be able to act through both pathways to control cell proliferation. Alternately, if the Rb/p53 combination therapeutic agent were introduced into a cell having a mutation that rendered the cell resistant to one of the tumor suppressors, the other tumor suppressor may nonetheless be able to function.

In accordance yet with another aspect of the invention, the therapeutic agent contains telomerase (TRT) and a combination of both Rb and p53, or nucleotide sequences encoding these proteins. It is known that p53 and Rb regulate cell proliferation through different pathways, such that a therapeutic agent having telomerase (TRT) with a combination of Rb and p53 would be able to act through both pathways to control cell proliferation. Alternately, if the telomerase/Rb/p53 combination agent were introduced into a cell having a mutation that rendered the cell resistant to one of the tumor suppressors, the other tumor suppressor may nonetheless be able to function.

Other Compounds that Regulate Cell Proliferation

Suitable tumor suppressors for use in therapeutic agents directed to inhibiting cell senescence in accordance with the present invention further include, but are not limited to, one or more of the following: other members of the p53 family including p63 or p73 as disclosed by Flores et al. (2002, Nature 416:560-564); members of the p62 and p160 family of polypeptides that affect proliferation, aggregation, differentiation and survival of leukocytes, and inhibit ubiquitination of p53, disclosed in U.S. Pat. No. 6,291,645; ETS2 Repressor Factor (ERF) transcriptional repressor from the ets oncogene family, especially the use of ERF and ERF chimeric molecules and fusion proteins to reduce ets-dependent tumorigenicity in a tumor cell, disclosed in U.S. Pat. Nos. 6,194,547 and 5,856,125; members of the BRCA family, including the normal gene products of BRCA1 or BRCA2 which are tumor suppressors as disclosed in U.S. Pat. No. 6,149,903; tumor suppressor C4-2 proteins found in high levels in normal brain tissue and in very low levels in several brain tumors, disclosed in U.S. Pat. No. 5,990,294; HIV-derived polypeptide(s) disclosed in U.S. Pat. No. 6,316,210, where expression of the polynucleotide encoding certain HIV-derived polypeptides inhibits hdm2 translocation; the “large tumor suppressor” or lats, disclosed in U.S. Pat. No. 6,359,193; the p202 tumor suppressor disclosed in U.S. Pat. No. 6,331,284, especially to inhibit development of the transformation phenotype and tumorigenicity of breast cancer cells, prostate cancer cells, and pancreatic cancer cells; E6-targeted protein 1 (E6TP1), a GAP protein that binds to E6 protein of human papillomavirus, useful in regulation of small G-protein signalling pathways and control of cell proliferation, disclosed in U.S. Pat. No. 6,440,696; an adenomatous polyposis coli (APC) tumor suppressor or “gatekeeping” protein involved in regulation of colorectal tumorigenesis, disclosed in U.S. Pat. No. 5,998,600; a 14 kDa protein with the ability to inhibit endothelial cell proliferation in vitro, disclosed in U.S. Pat. No. 5,854,221; mutated E2F protein, in particular mutated E2F1, that produces dominant interfering mutants that act as tumor suppressors, disclosed in U.S. Pat. No. 5,869,040; mannose 6-phosphate/insulin-like growth factor-II (M6P/IGF-II) receptor as a tumor suppressor, disclosed in U.S. Pat. No. 5,874,222; maspin, a serine protease inhibitor, disclosed in U.S. Pat. No. 5,905,023; a p53-derived peptide having the ability to bind to a human MHC Class I molecule, disclosed in U.S. Pat. No. 5,679,641; or members of a family of cell-cycle regulatory (CCR) proteins including p13.5, p15, and p16, disclosed in U.S. Pat. No. 6,486,131. It is understood that a therapeutic agent in accordance with the present invention may include a protein having tumor suppressor activity or may include the nucleic acid encoding the protein. It is understood that for a particular embodiment, one of skill in the art can prepare a therapeutic agent that includes the tumor suppressor protein or the nucleic acid encoding the protein, by using well-known methods in combination with the teachings of references disclosing the tumor suppressor(s) of interest in a particular embodiment.

If desired, a composition of the present invention may include a transport agent capable of directing a therapeutic agent to a specific intracellular location, as disclosed above. In one embodiment, a transport agent involved in intracellular targeting is a nuclear localization signal involved in delivering a therapeutic agent to the nucleus.

### PREPARATION OF COMPOSITIONS OF THE PRESENT INVENTION

Compositions of the present invention can be prepared by chemical conjugation of components to form chimeric molecules. Alternately, compositions of the present invention can be prepared as fusion proteins encoded by nucleic acid constructs having at least two adjacent nucleotide sequences that are not found adjacent in nature. Alternately, such nucleic acid constructs can be made so as to include spacer sequences in the resulting fusion protein that enhance the ability of the effective protein portions to assume desired and effective configurations without undesirable structural influence from nearby proteins or fragments thereof.

Chemical Conjugation.

Polypeptides can be chemically conjugated by means well known to those of skill in the art. The procedure for attaching one polypeptide to another varies according to the chemical structure of each polypeptide, e.g., as disclosed by U.S. Pat. No. 6,437,095. Polypeptides typically contain a variety of functional groups; e.g., carboxylic acid (—COOH) or free amine (—NH₂) groups, which are available for reaction with a suitable functional group on either polypeptide. Alternatively, polypeptides are derivatized to attach additional reactive functional groups. The derivatization optionally involves attachment of linker molecules such as those available from Pierce Chemical Company (Rockford Ill.). As used herein, a “linker” is a molecule that is used to join one polypeptide to another. One class of popular linkers are N-hydroxysuccinimide esters (NHS esters) that react with primary amines (especially lysine and amino termini). Because lysine residues are abundant on the surface of most proteins, these cross-linkers will bind efficiently to almost any protein. NHS ester reactions are generally carried out at pH 7.0-9.0 Reactivity of the lysine group increases as the pH increases to 9.0, but the competing NHS hydrolysis reaction is also favored with pH increase.

In accordance with one aspect of the present invention, transport agents can be chemically conjugated to therapeutic agents as disclosed elsewhere herein. In accordance with another aspect of the invention, regions of therapeutic agents can be chemically conjugated.

In one embodiment, a linker is used to join telomerase or TRT to p53 protein. In another embodiment, a linker is used to join telomerase or TRT to Rb protein. The linker is capable of forming covalent bonds with both polypeptides. Suitable linkers are well known to those of skill in the art and include, but are not limited to, straight or branched-chain carbon linkers, heterocyclic carbon linkers, or peptide linkers. In particular, two polypeptides can be joined to the constituent amino acids through their side groups (e.g., through a disulfide linkage to cysteine), or to the alpha-carbon amino and carboxyl groups of the terminal amino acids. Alternately, a bifunctional linker having one functional group reactive with groups on each polypeptide can be used to form the desired conjugate. Alternatively, derivatization can proceed through chemical treatment of either or both polypeptides being joined, e.g., glycol cleavage of the sugar moiety of a glycoprotein with periodate to generate free aldehyde groups. The free aldehyde groups on the glycoprotein may be reacted with free amine or hydrazine groups on an agent to bind the agent thereto (U.S. Pat. No. 4,671,958). Procedures for generation of free sulfhydryl groups on polypeptides, are known (U.S. Pat. No. 4,659,839). Moreover, many procedures and linker molecules for attachment of various compounds to proteins are known. (U.S. Pat. Nos. 4,671,958; 4,659,839; 4,414,148; 4,699,784; 4,680,338; 4,569,789; and 4,589,071).

Fusion Proteins

Many of the compositions described herein can be expressed as fusion proteins in suitable host cells, then harvested from host cells and administered to a target, where the target may be a cell, a collection of cells, a tissue, an organ, an organism, or an individual in accordance with methods provided herein. The term ‘“fusion proteins” include terms such as “coupled proteins,” “coupling products,” “chimeric proteins,” and “fusion products.” Fusion proteins suitable for use in the present invention can be expressed from nucleic acid constructs containing nucleotide sequences encoding therapeutic agent regions, where the nucleotide sequences have been fused “in-frame” to permit accurate translation of DNA to RNA to protein.

Fusion proteins can be made and used by means of standard recombinant DNA techniques. If necessary, fusion proteins can be made and used using methods that are analogous to or readily adaptable from standard recombinant DNA techniques. Nucleic acid constructs encoding fusion proteins suitable for use in the present invention, contain at least two nucleotide sequences encoding therapeutic agent components. Nucleic acid constructs encoding fusion proteins can be prepared and manipulated using standard recombinant DNA techniques and readily available adaptations thereof. Constructs encoding fusion proteins are prepared such that one or more open reading frames are operably linked to a suitable promoter sequence to form part of an expression vector. Expression vectors may contain additional regulatory elements as desired for a particular embodiment. The expression vector will be used to drive expression of fusion proteins in suitable host cells according to standard recombinant DNA techniques. The expression vector can, for example, be a recombinant virus vector or a non-viral transfection vector. Expression vectors and methods for producing recombinant proteins using expression vectors are disclosed, e.g., in Sambrook et al., Molecular Cloning, 2^{nd} Ed., Cold Spring Harbor Laboratory, 1989. It is understood that, for any nucleotide sequence capable of being transcribed and translated to produce a functional polypeptide, the degeneracy of the genetic code results in a plurality of nucleotide sequences that encode the same polypeptide, and each nucleotide sequence of this plurality of nucleotide sequences is an embodiment of the present invention.

Nucleotide sequences encoding therapeutic agent regions can be linked to other nucleic acids using methods known in the art. Nucleotide sequences can be linked by use of restriction enzymes to generate blunt ends or sticky ends, where nucleotide sequences having blunt or sticky ends are cloned into sites in a nucleic acid construct in an “in-frame” orientation. Nucleotide sequences can be chemically coupled to form a nucleic acid construct. Nucleotide sequences encoding therapeutic agent regions can be linked to non-nucleic acid molecules such as polypeptides or polysaccharides. If desired, nucleotide sequences encoding therapeutic agent regions can be prepared with a polypeptide tail for coupling to other molecules. Nucleotide sequences encoding therapeutic agent regions can be coupled or fused with one or more transport agents; suitable transport agents are described elsewhere herein.

Fusion proteins described herein can be used according to the invention as compositions capable of being taken up by a target population of cells, so that the therapeutic agent has effects on one or more cellular processes, with the result that cell senescence is inhibited.

Variants and fragments of proteins can be generated and tested using methods known in the art. In the polypeptides of the invention, mutations of the constituent amino acid sequences can be incorporated in the fusion polypeptides and other coupled proteins. Included herein are proteins having mutated sequences such that they remain homologous to a protein having the corresponding parent sequence, where the homology may be in sequence, function, or antigenic character. Such mutations can preferably for example be mutations involving conservative amino acid changes, e.g., changes between amino acids of broadly similar molecular properties. For example, interchanges within the aliphatic group alanine, valine, leucine and isoleucine can be considered as conservative. Sometimes, substitution of glycine for one of these can also be considered conservative. Interchanges within the aliphatic group aspartate and glutamate can also be considered as conservative. Interchanges within the amide group asparagine and glutamine can also be considered as conservative. Interchanges within the hydroxy group serine and threonine can also be considered as conservative. Interchanges within the aromatic group phenylalanine, tyrosine and tryptophan can also be considered as conservative. Interchanges within the basic group lysine, arginine and histidine can also be considered conservative. Interchanges within the sulfur-containing group methionine and cysteine can also be considered conservative. Sometimes substitution within the group methionine and leucine can also be considered conservative. Preferred conservative substitution groups are aspartate-glutamate; asparagine-glutamine; valine-leucine-isoleucine; alanine-valine; phenylalanine-tyrosine; and lysine-arginine. In other respects, mutated sequences can comprise insertions such that the overall amino acid sequence is lengthened while the protein variant or fragment retains desired properties. Additionally, mutated sequences can comprise random or designed internal deletions that shorten the overall amino acid sequence while the protein variant or fragment retains desired properties.

The mutated protein sequences can additionally or alternatively be encoded by nucleotide sequences that hybridize under stringent conditions with the appropriate strand of the naturally-occurring nucleotide sequence encoding the parent protein, and can be tested for positive results in known functional tests relevant to the parent protein. “Stringent conditions” are sequence-dependent and will vary according to the circumstances of the hybridization reaction. Generally, stringent conditions can be selected to be about 5° C. lower than the thermal melting point (T_{M}) for the specific sequence at a defined ionic strength and pH. The T_{M} is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 60° C. Because other factors may also affect the stringency of hybridization including, inter alia, base composition and size of the complementary strands, the presence of organic solvents and the extent of base mismatching, it is understood that the combination of parameters is more important for determining stringent conditions than the absolute measure of any one.

The following examples are provided to illustrate the present invention, and are not intended in any way to limit the scope of the invention.

### EXAMPLES

### Example 1

### Chemically Conjugated Chimeric hTRT-p53 Protein

Recombinant Telomerase Reverse Transcriptase

As a first step in making and using a composition that includes a transport agent and a therapeutic agent that includes human telomerase reverse transcriptase (hTRT) and p53, large quantities of recombinant hTRT are prepared. Recombinant hTRT is prepared by a modification of the method described in U.S. Pat. No. 6,475,789. Briefly, a lambda cDNA library is derived from the human 293 cell line, which expresses high levels of telomerase activity, and the library is partitioned into 25 pools containing about 200,000 plaques each. Each pool is screened by polymerase chain reaction (PCR). Subpools of one positive primary pool are further screened by PCR using the same primer pair. For both the primary and the secondary subpool screening, hTRT sequence is amplified for a total of 31 cycles at 94° C. for 45 seconds, then 60° C. for 45 seconds, and then 72° C. for 90 seconds. One hTRT-positive subpool from the secondary screening is then screened by plaque hybridization with a probe from the 5′ region of clone #712562, an hTRT cDNA clone having all eight telomerase RT (TRT) motifs (Clone #712562 available from the I.M.A.G.E. Consortium at the Human Genome Center, DOE, Lawrence Livermore National Laboratory, derived from a cDNA library of germinal B cells derived by flow sorting of tonsil cells.) One phage is positively identified and it contains an approximately four kilobase (kb) insert that is excised and subcloned into the EcoRI site of pBluescript II SK+ vector (Stratagene) as an EcoRI fragment.

To produce large quantities of full-length hTRT, the bacterial expression vector pThioHis A (Invitrogen) is selected as an expression system. The hTRT-coding insert includes nucleotides 707 to 4776 of the hTRT insert in the plasmid pGRN121, and this nucleotide sequence includes the complete coding sequence for the hTRT protein. This expression vector is designed for inducible expression in bacteria, producing high levels of a fusion protein composed of a cleavable, HIS tagged thioredoxin moiety and the full length hTRT protein in E. coli. The expression system is used substantially in accordance with the manufacturer's instructions. Full length recombinant hTRT is expressed, purified, and the HIS tag is removed.

Telomerase Assay

Telomerase activity is assayed by a modification of a method disclosed in U.S. Pat. No. 6,300,131. Cell extracts, immunoprecipitation supernatants, or pellet fractions are assayed in a two step telomerase assay (TRAP) similar to that previously described by Autexier et al. (1996, EMBO J., 15:5928-5935). This two step procedure uses a limited number of PCR cycles for amplification of the telomerase products so that the signal will be in the linear range, producing relative signal intensities that reflect relative activity in a semi-quantitative manner. Negative controls either have no extract or are RNase treated. An internal standard for product amplification in the PCR step of the assay is included in each reaction.

Recombinant p53 Protein

As a next step in making and using a chimeric composition that includes a transport agent, human telomerase reverse transcriptase (hTRT) and p53, large quantities of recombinant p53 are prepared. Recombinant p53 protein is prepared by a modification of the method of Muller et al., 1998, Proc Natl Acad Sci USA 95:6079-6084. Recombinant p53 cDNA is cloned into the bacterial expression vector pT5T. Bacterial cultures are grown at 30° C. and expression is induced with 1 mM isopropyl-D-thiogalactopyranoside (IPTG). Bacteria are harvested by centrifugation 4 hr after induction. The bacterial pellet is resuspended in glycerol with 0.7% Triton X-100 and 0.4% 2-mercaptoethanol. Bacteria are lysed in extraction buffer containing 10 mM Tris-HCl, pH 8.0, 500 mM NaCl, 5 mM EDTA, 1 mM DTT, 0.1 mM ZnOac, and 6 mg/ml lysozyme/complete protease inhibitors (Boehringer Mannheim). Bacterial DNA is degraded by addition of 50 μg/ml of DNase I. The suspension is cleared by centrifugation at 120,000×g at 4° C. Supernatant is stored at 80° C. Primary anti-p53 mAb PAb240 is used to immunoprecipitate p53 (Oncogene Science).

Conjugation of hTRT and p53

Chemical conjugation of hTRT protein and p53 protein, prepared as described above, is carried out using dimethyl suberimidate-2HCl, a longer-chain, water-soluble, membrane permeable, imidoester cross-linker (Pierce Biotechnology, Product No. 20700) according to manufacturer's instructions. Chimeric proteins are isolated by gel filtration chromatography.

Linking Transport Agent to hTRT-p53 Protein

In order to introduce the chimeric TRT-53 protein into cell, a translocation peptide that naturally conveys peptides across both cellular and the nuclear membranes is linked to the chimeric protein. Penetratin (Oncor) is used in according to manufacturer's instructions to couple Penetratin to chimeric TRT-p53 by means of a disulfide bond through cysteine residues at the end of each peptide. Unbound Penetratin is separated from higher-molecular-weight chimeric protein by ultrafiltration.

Measurement of Transmembrane Transport of Penetratin-hTRT-p53 Protein

An aliquot of penetratin-labelled-chimeric TRT-p53 protein is labelled with fluorescein (EZ-Label Fluorescein Protein Labelling Kit, Pierce Biotechnology, catalog no. 53000) according to manufacturer's instructions. Target cells in suspension (10⁶ cells/ml) are incubated with fluorescein-labelled proteins at 37° C. in PBS pH 7.2 containing 2% fetal calf serum (PBS/FCS) in 96-well plates. After incubation for 15 minutes, cells are pelleted by centrifugation, washed three times with PBS/FCS containing 1% sodium azide, incubated with trypsin/EDTA (Gibco) at 37° C. for five minutes, then washed twice more with PBS/FCS/NaN₃. Pelleted cells are resuspended in PBS containing 2% FCS and 0.1% propidium iodide and analyzed on a FACScan (Becton Dickenson). Cells identified by flow cytometry (FACScan) as containing fluorescently-labelled proteins are isolated, cultured, and the presence of chimeric TRT-p53 protein inside these cells is confirmed by immunoblotting using anti-TRT and anti-p53 antibodies.

### Example 2

### VP22-Telomerase-p53 Fusion Protein

Sequence encoding human TRT (having telomerase activity) is fused in frame with sequences encoding VP22 (as a transport agent) and normal p53 (as a tumor suppressor) to form a VP22-telomerase-p53 fusion protein, by a modification of methods disclosed in Phelan et al. (1998, Nature Biotechnology 16:440-443) and U.S. Pat. No. 6,358,739.

VP22-p53 Fusion Construct.

The coding region for normal p53 is amplified by PCR using primers that contain either BglII or BamHI sites. Plasmid pc49epB contains the VP22 open reading frame in the background of pcDNAamp 1.1 (Invitrogen), with cytomegalovirus (CMV) promoter and N-terminal region derived from pGE109, such that the ATG start codon is immediately preceded by a unique BglII site. In the C-terminus of VP22 derived from plasmid pUL49ep, the last residue is immediately preceded by a unique BamHI site and reads in frame to an epitope tag sequence recognized by monoclonal antibody CMV-018-48151. Coding region for normal p53 is amplified by PCR using primers that contain BglII sites, and the PCR product is cloned into the unique BglII site such that VP22, p53, and the epitope tag remain in frame for protein expression.

VP22-Telomerase-p53 Fusion Construct.

Oligonucleotides are custom synthesized for use as PCR primers for cloning human telomerase reverse transcriptase (hTRT) cDNA, where primers as disclosed in U.S. Pat. No. 6,358,739 (Life-Technologies or Microsynth) are modified to contain BamHI sites in frame with the coding region for hTRT. Reverse-transcriptase-polymerase chain reaction (RT-PCR) is carried out using cDNA prepared from 293T cells, Taq polymerase, and the custom synthesized primers disclosed above, to produce a 3417 base-pair product. The PCR product is cloned into the unique BamHI site in the VP22-p53 fusion construct, such that the hTRT coding region is in frame with the coding region for VP22, p53 protein, and the epitope tag for protein expression. Nucleotide sequence determination of the resulting clone demonstrates that the cloned hTRT sequence has the correct nucleotide sequence when compared to published hTRT nucleotide sequence in GenBank Accession Number AF015950.

Expression of Fusion Protein.

COS cells are plated at 2×10⁵ cells per 35 mm dish and transfected with 1 μg of VP22-hTRT-p53 expression plasmid made up to 2 μg with pUC19DNA, using the calcium phosphate precipitation technique modified with BES-buffered saline. Monoclonal antibodies are used to detect expression of the fusion protein, including anti-p53 antibody, antiVP22 antibody, and mAb CMV-018-48151 against the epitope tag.

Treating Cells with VP22-hTRT-p53 Fusion Protein.

Medium containing fusion protein and COS cells expressing fusion protein are collected and cells are lysed to release contents. Fusion protein is immunoprecipitated using mAB CMV-018-48151 against the epitope tag. MDX1 primary human fibroblasts are incubated with medium containing immunoprecipitated fusion protein. Intracellular localization of fusion proteins is detected using anti-VP22 and anti-p53 protein antibodies and indirect immunofluorescence staining. MDXI cells treated with VP22-hTRT-p53 fusion protein show intense staining in the nucleus, and this pattern is seen using anti-VP22 and anti-p53 protein antibodies.

Telomerase Assay.

The Telomeric Repeat Amplification Protocol (TRAP) assay is performed according to the manufacturer's protocol (TRAPeze Telomerase Detection Kit, Oncor). Briefly, a pellet of 50,000 cells treated with the VP22-hTRT-p53 fusion protein is resuspended in 50 l of CHAP lysis buffer (1×) containing RnaseOut at 200 U/ml (Gibco Life-Technology). The cell suspension is incubated on ice for 30 minutes and immediately centrifuged at 15,000 RPM at 4° C. for 15 minutes. The supernatant is immediately transferred to an RNase-free tube. According to the manufacturer's protocol, the cell extract is diluted 1:10 and a cell extract from 200 cells is used for the TRAP assay. Ten microliters (μl) of the reaction mix are resolved via 12.5% non-denaturing polyacrylamide gel electrophoresis (PAGE) in TBE buffer (0.5×) at 150 volts for 2 hours. The DNA ladders are visualized by staining with SYBR Green Stain (Molecular Probe). Normal MDX1 primary human fibroblasts do not possess detectable telomerase enzyme activity as monitored by the TRAP assay. Thus, any detected telomerase enzyme activity from the MDX1 cells exposed to VP22-hTRT-p53 fusion protein can be attributed to the fusion proteins that are taken up by the primary human fibroblast MDX1 cells. PAGE results show telomerase activity in MDX1 cells exposed to the VP22-hTRT-p53 fusion protein, by showing positive ladder formation in cells exposed to the fusion protein.

Demonstration of Enhanced Proliferation Capacity.

A population of MDX1 cells are treated with VP22-hTRT-p53 fusion protein. Another population of MDX1 cells are treated with denatured VP22-hTRT-p53 fusion protein. Another population of MDX1 is not exposed to fusion protein. MDX1 cells treated with intact VP22-hTRT-p53 fusion protein show an enhanced population doubling curve compared to cells treated with denatured fusion protein and untreated cells.

Demonstration of the Catalytic Enzyme Activity by VP22-hTRT-p53 Fusion Proteins.

To demonstrate that the VP22 and p53 protein fusions do not affect the catalytic activity of the hTRT enzyme, TRAP enzyme assays are performed on total cell extracts from telomerase-negative MDX1 cells incubated with VP22-hTRT-p53 fusion protein. Cells extracts incubated with fusion proteins show clear ladder formation, indicating the preservation of the telomerase catalytic activity in the VP22-hTRT-p53 fusion proteins.

The foregoing descriptions and Examples illustrate selected embodiments of the present invention and in light thereof various modifications will be suggested to one of skill in the art, all of which are in the spirit and purview of this invention.

What is claimed is:

1. A composition for inhibiting cell senescence comprising a transport agent and a therapeutic agent, wherein the therapeutic agent comprises a first region having telomerase activity and a second region having tumor suppressor activity.

2. A composition of claim 1 further comprising a cleavage site that is cleavable in vivo.

3. A composition of claim 2 wherein the cleavage site is located between the transport agent and the therapeutic agent.

4. A composition of claim 3, wherein the cleavage site is a polypeptide.

5. A composition of claim 1 wherein the transport agent comprises a polypeptide.

6. A composition of claim 5 wherein the transport agent comprises an arginine-rich polypeptide.

7. A composition of claim 5 wherein the transport agent comprises a trans-activating transduction (tat) polypeptide.

8. A composition of claim 7 wherein the transport agent is derived from an HIV tat protein.

9. A composition of claim 8 wherein the transport agent comprises amino acid residues 48-60 of HIV tat protein.

10. A composition of claim 1 wherein the first region of the therapeutic agent comprises telomerase ribonucleoprotein or a therapeutically effective fragment thereof.

11. A composition of claim 1 wherein the first region of the therapeutic agent comprises telomerase reverse transcriptase (TRT) or a therapeutically effective fragment thereof.

12. A composition of claim 1 wherein the second region of the therapeutic agent comprises at least one polypeptide having normal p53 protein activity.

13. A composition of claim 1 wherein the second region of the therapeutic agent comprises at least one polypeptide having normal retinoblastoma (Rb) protein activity.

14. A composition of claim 1 wherein the therapeutic agent comprises TRT or a therapeutically effective fragment thereof, and a polypeptide having normal p53 protein activity.

15. A composition of claim 1 wherein the therapeutic agent comprises TRT or a therapeutically effective fragment thereof and a polypeptide having normal RB protein activity.

16. A composition of claim 1 wherein the therapeutic agent comprises TRT or a therapeutically effective fragment thereof, a polypeptide having normal p53 protein activity, and a polypeptide having normal Rb protein activity.

17. A composition of claim 1 wherein the second region of the therapeutic agent comprises at least one polypeptide selected from the group consisting of: p53 protein; retinoblastoma (Rb) protein; p62 protein; p160 protein; ETS2 repressor factor (ERF); BRCA protein; C4-2 protein; HIV-derived polypeptide; “large tumor suppressor” (lats) protein; p202 protein; E6-targeted protein 1 (E6TP1); adenomatous polyposis coli (APC) tumor suppressor protein; 14 kDa protein with the ability to inhibit endothelial cell proliferation in vitro; mutated E2F protein; mannose 6-phosphate/insulin-like growth factor-II (M6P/IGF-II) receptor; maspin; or cell-cycle regulatory (CCR) tumor suppressor protein.

18. A composition of claim 1, wherein the second region of the therapeutic agent comprises at least one p53 protein selected from the group consisting of: normal 53 protein or an effective fragment or variant thereof; p53 protein mutated to remain in active form; p53 isoform with a C-terminal region removed; normal p63 protein; or normal p73 protein.

19. A composition of claim 18 wherein the second region of the therapeutic agent comprises normal p53 protein and normal p63 protein.

20. A composition of claim 18 wherein the second region of the therapeutic agent comprises normal p53 protein and normal p73 protein.

21. A composition of claim 18 wherein the second region comprises normal p53 protein, normal p63 protein, and normal p73 protein.

22. A composition of claim 1 wherein the transport agent and the therapeutic agent are chemically conjugated.

23. A composition of claim 22 wherein the first region and the second region of the therapeutic agent are chemically conjugated.

24. A composition of claim 1 comprising a fusion protein.

25. A composition of claim 24 wherein the therapeutic agent is the fusion protein and the transport agent is chemically conjugated to the therapeutic agent.

26. A cell comprising a composition of claim 1.

27. A cell of claim 26 wherein the cell is an adult stem cell.

28. A nucleotide sequence encoding a fusion protein comprising a transport agent and a therapeutic agent comprising a first region having telomerase activity and a second region having tumor suppressor activity.

29. An expression vector comprising the nucleotide sequence of claim 28.

30. A cell comprising an expression vector of claim 29.

31. A pharmaceutical formulation comprising a pharmaceutically acceptable excipient and a composition capable of inhibiting cell senescence, wherein the composition comprises a transport agent and a therapeutic agent, the therapeutic agent comprising a first region having telomerase activity and a second region having tumor suppressor activity.

32. A pharmaceutical formulation of claim 31 comprising a transport agent and a protein therapeutic agent, wherein the therapeutic agent comprises a first region having telomerase activity and a second region having tumor suppressor activity.

33. A pharmaceutical formulation of claim 31 comprising a nucleotide sequence encoding a fusion protein comprising a transport agent and a therapeutic agent, wherein the therapeutic agent comprises a first region having telomerase activity and a second region having tumor suppressor activity.

34. A method of inhibiting cell senescence comprising contacting a cell with an effective amount of a composition comprising a transport agent and a therapeutic agent, wherein the therapeutic agent comprises a first region having telomerase activity and a second region having tumor suppressor activity.

35. The method of Claim,34 wherein the cell is part of a collection of cells, a tissue, an organ, an organism, or an individual.

36. The method of claim 35, wherein contacting the cell that is part of a collection of cells, a tissue, an organ, an organism, or an individual comprises contacting each cell of the collection of cells, tissue, organ, organism, or individual.

37. The method of claim 34 wherein the cell is an adult stem cell.

38. A method for inhibiting cell senescence in an organism or an individual comprising:
(a) providing a composition comprising a transport agent and a therapeutic agent, wherein the therapeutic agent comprises a first region having telomerase activity and a second region having tumor suppressor activity;
(b) administering to the organism or individual in need thereof an amount of the composition sufficient to inhibit cell senescence.

39. The method of claim 38, wherein the composition is administered ex vivo.

40. The method of claim 38, wherein the composition is administered in vivo.

41. The method of claim 40, wherein the composition is administered intramuscularly, intradermally, or subcutaneously.

42. A method of extending the lifespan of an organism comprising:
(a) providing a composition comprising a transport agent and a therapeutic agent, wherein the therapeutic agent comprises a first region having telomerase activity and a second region having tumor suppressor activity;
(b) administering to the organism an amount of the composition sufficient to extend the lifespan of the organism.

43. A method of extending the lifespan of an individual comprising:
(a) providing a composition comprising a transport agent and a therapeutic agent, wherein the therapeutic agent comprises a first region having telomerase activity and a second region having tumor suppressor activity;
(b) administering to the individual an amount of the composition sufficient to extend the lifespan of the individual.

44. A composition for inhibiting undesirable cell proliferation comprising a transport agent and a therapeutic agent, wherein the therapeutic agent comprises a first region having telomerase activity and a second region having tumor suppressor activity.

45. A composition of claim 44, wherein the undesirable cell proliferation is cancer.

46. A composition of claim 44, wherein the undesirable cell proliferation is a non-cancerous hyperproliferative disorder.

47. A composition of claim 46, wherein the hyperproliferative disorder is macular degeneration.

48. A composition of claim 46, wherein the hyperproliferative disorder is diabetic retinopathy.

49. A composition of claim 44 further comprising a cleavage site that is cleavable in vivo.

50. A composition of claim 49 wherein the cleavage site is located between the transport agent and the therapeutic agent.

51. A composition of claim 50, wherein the cleavage site is a polypeptide.

52. A composition of claim 44 wherein the transport agent comprises a polypeptide.

53. A composition of claim 52 wherein the transport agent comprises an arginine-rich polypeptide.

54. A composition of claim 52 wherein the transport agent comprises a trans-activating transduction (tat) polypeptide.

55. A composition of claim 54 wherein the transport agent is derived from an HIV tat protein.

56. A composition of claim 55 wherein the transport agent comprises amino acid residues 48-60 of HIV tat protein.

57. A composition of claim 44 wherein the first region of the therapeutic agent comprises telomerase ribonucleoprotein or a therapeutically effective fragment thereof.

58. A composition of claim 44 wherein the first region of the therapeutic agent comprises telomerase reverse transcriptase (TRT) or a therapeutically effective fragment thereof.

59. A composition of claim 44 wherein the second region of the therapeutic agent comprises at least one polypeptide having normal p53 protein activity.

60. A composition of claim 44 wherein the second region of the therapeutic agent comprises at least one polypeptide having normal retinoblastoma (Rb) protein activity.

61. A composition of claim 44 wherein the therapeutic agent comprises TRT or a therapeutically effective fragment thereof, and a polypeptide having normal p53 protein activity.

62. A composition of claim 44 wherein the therapeutic agent comprises TRT or a therapeutically effective fragment thereof, and a polypeptide having normal RB protein activity.

63. A composition of claim 44 wherein the therapeutic agent comprises TRT or a therapeutically effective fragment thereof, a polypeptide having normal p53 protein activity, and a polypeptide having normal Rb protein activity.

64. A composition of claim 44 wherein the second region of the therapeutic agent comprises at least one polypeptide selected from the group consisting of: p53 protein; retinoblastoma (Rb) protein; p62 protein; p160 protein; ETS2 repressor factor (ERF); BRCA protein; C4-2 protein; HIV-derived polypeptide; “large tumor suppressor” (lats) protein; p202 protein; E6-targeted protein 1 (E6TP1); adenomatous polyposis coli (APC) tumor suppressor protein; 14 kDa protein with the ability to inhibit endothelial cell proliferation in vitro; mutated E2F protein; mannose 6-phosphate/insulin-like growth factor-II (M6P/IGF-II) receptor; maspin; or cell-cycle regulatory (CCR) tumor suppressor protein.

65. A composition of claim 44, wherein the second region of the therapeutic agent comprises at least one p53 protein selected from the group consisting of: normal 53 protein or a therapeutically effective fragment or variant thereof; p53 protein mutated to remain in active form; p53 isoform with a C-terminal region removed; normal p63 protein; or normal p73 protein.

66. A composition of claim 65 wherein the second region of the therapeutic agent comprises normal p53 protein and normal p63 protein.

67. A composition of claim 65 wherein the second region of the therapeutic agent comprises normal p53 protein and normal p73 protein.

68. A composition of claim 65 wherein the second region comprises normal p53 protein, normal p63 protein, and normal p73 protein.

69. A composition of claim 44 wherein the transport agent and the therapeutic agent are chemically conjugated.

70. A composition of claim 69 wherein the first region and the second region of the therapeutic agent are chemically conjugated.

71. A composition of claim 44 comprising a fusion protein.

72. A composition of claim 71 wherein the therapeutic agent is a fusion protein and the transport agent is chemically conjugated to the therapeutic agent.

73. A cell comprising a composition of claim 44.

74. A cell of claim 73 wherein the cell is an adult stem cell.

75. A pharmaceutical formulation comprising a pharmaceutically acceptable excipient and a composition capable of inhibiting undesirable cell proliferation, wherein the composition comprises a transport agent and a therapeutic agent, the therapeutic agent comprising a first region having telomerase activity and a second region having tumor suppressor activity.

76. A pharmaceutical formulation of claim 75 comprising a transport agent and a protein therapeutic agent, wherein the therapeutic agent comprises a first region having telomerase activity and a second region having tumor suppressor activity.

77. A pharmaceutical formulation of claim 75 comprising a nucleotide sequence encoding a fusion protein comprising a transport agent and a therapeutic agent, wherein the therapeutic agent comprises a first region having telomerase activity and a second region having tumor suppressor activity.

78. A composition of claim 75, wherein the undesirable cell proliferation is cancer.

79. A composition of claim 75, wherein the undesirable cell proliferation is a non-cancerous hyperproliferative disorder.

80. A composition of claim 79, wherein the hyperproliferative disorder is macular degeneration.

81. A composition of claim 79, wherein the hyperproliferative disorder is diabetic retinopathy.

82. A method for inhibiting undesirable cell proliferation in an organism comprising:
(a) providing a composition comprising a transport agent and a therapeutic agent, wherein the therapeutic agent comprises a first region having telomerase activity and a second region having tumor suppressor activity;
(b) administering to the organism an amount of the composition sufficient to inhibit undesirable cell proliferation.

83. The method of claim 82, wherein the undesirable cell proliferation is cancer.

84. The method of claim 82, wherein the undesirable cell proliferation is a non-cancerous hyperproliferative disorder.

85. A method for inhibiting undesirable cell proliferation in an individual comprising:
(a) providing a composition comprising a transport agent and a therapeutic agent, wherein the therapeutic agent comprises a first region having telomerase activity and a second region having tumor suppressor activity;
(b) administering to the individual an amount of the composition sufficient to inhibit undesirable cell proliferation.

86. The method of claim 85, wherein the undesirable cell proliferation is cancer.

87. The method of claim 85, wherein the undesirable cell proliferation is a non-cancerous hyperproliferative disorder.
